(19) United States
(12) Patent Application Publication
(10) Pub. No.: US 2025/0242349 A1
(43) Pub. Date: Jul. 31, 2025

(54) MICROFLUIDIC DETECTION DEVICES

(71) Applicants: Research & Business Foundation Sungkyunkwan University, Suwon-si (KR); Ninebiowear Co., Ltd., Suwon-si (KR)

(72) Inventors: Nae-Eung LEE, Seoul (KR); Sandeep KAUSHAL, Suwon-si (KR)

(21) Appl. No.: 19/042,486

(22) Filed: Jan. 31, 2025

(30) Foreign Application Priority Data
Jan. 31, 2024 (KR) 10-2024-0014886

(51) Int. Cl. B01L 3/00

(52) U.S. Cl. CPC B01L 3/502761; B01L 3/5023; B01L 2200/0668; B01L 2200/16; B01L 2300/048; B01L 2300/069; B01L 2300/0883; B01L 2400/0406; B01L 2400/0622

(57) ABSTRACT

Proposed is a microfluidic detection device in which microfluid flows by a capillary phenomenon. The microfluidic detection device includes an upper layer provided with a first inlet part configured to supply a fluid sample, and a middle layer disposed below the upper layer and provided with a second inlet part interlocked with the first inlet part, the middle layer being provided with a plurality of channels forming at least one complex by coupling the fluid sample to each probe. Furthermore, the microfluidic detection device includes a lower layer disposed below the middle layer and provided with a magnet seating part supported by the middle layer and a magnet.

### BACKGROUND

### Technical Field

The present disclosure relates to a microfluidic detection device. More particularly, the present disclosure relates to a microfluidic detection device configured such that a first complex is formed by coupling a fluid sample containing a bio-marker to a detection probe, a second complex is formed by coupling the first complex to a capture probe containing a magnetic bead, a sensing part having a magnet attached to a lower end of the sensing part concentrates the second complex by the magnetic bead, and the bio-marker of the fluid sample is detected in the sensing part.

### Description of the Related Art

A microfluidic detection device is used in various applications fields, and is particularly used as a key tool in life science and medical diagnosis in modern medical and life science research. The microfluidic detection device is mainly used for rapidly analyzing and diagnosing a fluid sample including a bio-marker.

Particularly, the microfluidic detection device has an advantage that the microfluidic detection device is capable of performing analysis with a small amount of a sample, and that the microfluidic fluid detection device is capable of detecting a fluid sample with high sensitivity and specificity within a fast time.

However, there is a problem that a conventional microfluidic detection device requires a manual processing of a bio-sample, an addition of a reagent, and a plurality of washing processes, so that there is a high probability that erroneous data is obtained due to an increase in complexity of the analysis process and an increase in worker interference.

In addition, in a passive microfluidic detection device, since the passive microfluidic detection device is vulnerable to a structure that eliminates bubble generation that hinders the flow of the fluid, the passive microfluidic detection device destabilizes the flow of the fluid, reduces the flow speed, and increases the pressure of the fluid, so that the performance of the passive microfluidic detection device is reduced and a situation where detection is impossible may occur.

In addition, since a probe fixing function for fixing a probe, the technology of cleaning a biomolecule that is not coupled to the probe, and the technology of removing the biomolecule that is not coupled to the probe required for accurate data reading are required to be combined, there is a problem that a stable operation is difficult to be realized.

Accordingly, in order to stably output data from a fluid sample, a method for simplifying processing of the fluid sample to minimize worker operations and ensure easy device operation.

In addition, an integrated device capable of smoothly moving a fluid sample by a non-invasive method within a microfluidic detection device, fixing and coupling a probe to the fluid sample for an immunoassay reaction, and efficiently detecting a bio-marker of the fluid sample by removing and washing an uncoupled biomolecule is required.

### SUMMARY

Accordingly, the present disclosure has been made keeping in mind the above problems occurring in the related art, and an objective of the present disclosure is to provide a microfluidic detection device configured such that a first complex is formed by coupling a fluid sample containing a bio-marker to a detection probe, that a second complex is formed by coupling the first complex to a capture probe containing a magnetic bead, that a sensing part having a magnet attached to a lower end of the sensing part concentrates the second complex by the magnetic bead, and that the bio-marker of the fluid sample is detected in the sensing part.

The technical problem to be solved by the present disclosure is not limited to the above-mentioned problem, and other problems which are not mentioned will be clearly understood by those skilled in the art from the following description.

In order to achieve the objective of the present disclosure, according to an aspect of the present disclosure, there is provided a microfluidic detection device in which microfluid flows by a capillary phenomenon, the microfluidic detection device including: an upper layer provided with a first inlet part configured to supply a fluid sample; a middle layer disposed below the upper layer and provided with a second inlet part interlocked with the first inlet part, the middle layer being provided with a plurality of channels forming at least one complex by coupling the fluid sample to each probe; and a lower layer disposed below the middle layer and provided with a magnet seating part supported by the middle layer and a magnet.

According to an aspect of the present disclosure, the microfluidic detection device may further include a first glass fiber, a second glass fiber, and an absorption pad that are disposed between the upper layer and the middle layer. Furthermore, a detection probe formed by coupling the probe to a label may be loaded in the first glass fiber, and a capture probe formed by coupling the probe to a magnetic bead may be loaded in the second glass fiber.

According to an aspect of the present disclosure, the upper layer may include: a pouch part filled with a washing solution; an opening and closing part connected to the pouch part and configured to supply the washing solution; a bubble collecting part configured to capture bubbles contained in the complex; a valve part provided on a first side of the bubble collecting part and configured to stop a flow of the washing solution for a predetermined time; and a first absorption part connected to the valve part and provided in a shape that surrounds the absorption pad, thereby absorbing a waste solution. Furthermore, the bubble collecting part may have a polygonal shape including a circular shape, and may be provided in a shape in which a height thereof decreases from an outer portion of the polygonal shape to a center of the polygonal shape, thereby being capable of collecting the bubbles.

According to an aspect of the present disclosure, the middle layer may include: a transferring part including a plurality of transferring parts provided so as to transfer the fluid sample and the complex; a first loading part in which the first glass fiber is seated, the first loading part being configured to load the detection probe; a first channel part in which a first complex is formed by coupling a bio-marker molecule contained in the fluid sample to the detection probe; a second loading part in which the second glass fiber is seated, the second loading part being configured to load the capture probe; a second channel part in which a second complex is formed by coupling the first complex to the capture probe; a sensing part configured to concentrate the magnetic bead contained in the second complex; and a second absorption part formed in a concave shape such that the absorption pad is seated therein, the second absorption part being configured to absorb the waste solution.

According to an aspect of the present disclosure, the first channel part may include: a first coupling region including a plurality of first coupling regions so as to maintain a latent time for mixing the first complex; and a first acceleration region including a plurality of first acceleration regions provided as expanded regions in the first coupling regions, the first acceleration region being configured to provide a diffusion time to the bio-marker molecule and the detection body contained in the fluid sample of the first complex.

According to an aspect of the present disclosure, the second loading part may include: a glass fiber seating region formed in a shape corresponding to that of the second glass fiber and in which the second glass fiber is seated; an expansion region provided on a first side of the glass fiber seating region and configured to control a flow of the first complex; and a mixing region provided on a second side of the glass fiber seating region and configured to accelerate mixing of the first complex and the capture probe.

According to an aspect of the present disclosure, the second channel part may include: a second coupling region including a plurality of second coupling regions so as to maintain a latent time for forming the second complex; and a second acceleration region including a plurality of second acceleration regions provided as expanded regions in the second coupling region, the second acceleration region being configured to provide a diffusion time to the first complex and the capture probe so that the second complex is formed.

According to an aspect of the present disclosure, the sensing part may include: a sensing region configured to concentrate and sense the magnetic bead contained in the second complex by being in contact with the magnet provided below the sensing region; and a washing region provided slantly such that the washing region is widened upward from the sensing region, the washing region being configured to provide a resistance force to the magnetic bead so that the magnetic bead is prevented from being washed out.

According to an aspect of the present disclosure, the sensing region may be configured to detect a bio-marker contained in the second complex.

According to an aspect of the present disclosure, the transferring part may include: a first flow path connecting the second inlet part and the first loading part to each other; a second flow path connecting the first loading part and the first channel part to each other; a third flow path connecting the first channel part and the second loading part to each other; a fourth flow path connecting the second loading part and the second channel part to each other; and a fifth transferring path connecting the second channel part and the sensing part to each other.

According to an aspect of the present disclosure, the transferring part may be configured to transfer the fluid sample and at least one of the complex by the capillary phenomenon.

According to an aspect of the present disclosure, an absorption region in which the absorption pad is accommodated may be formed by bonding the first absorption part and the second absorption part to each other.

According to an aspect of the present disclosure, the absorption region may be configured to absorb the washing solution so that the washing solution is prevented from backflowing to the sensing part.

According to an aspect of the present disclosure, the microfluidic detection device is a device structurally provided with a space for collecting bubbles that interfere with the flow of the fluid, forms the first complex by coupling the fluid sample to the detection probe, forms the second complex by coupling the first complex to the capture probe, and detects the bio-marker by concentrating the second complex containing the magnetic bead with the magnet. In the microfluidic detection device, as the fluid sample is moved by the capillary phenomenon, the microfluidic detection device is capable of being driven without an external power supply device, and the bio-marker is capable of being effectively detected by washing impurities that are not coupled to the second complex.

The effects of the present disclosure are not limited thereto and it should be understood that the effects include all effects that can be inferred from the configuration of the present disclosure described in the following specification or claims.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1 is a perspective view illustrating a microfluidic detection device according to an embodiment of the present disclosure;

FIG. 2 is an exploded perspective view illustrating the microfluidic detection device according to an embodiment of the present disclosure;

FIG. 3 is a perspective view illustrating an upper layer provided in the microfluidic detection device in FIG. 1;

FIG. 4 is a perspective view illustrating a middle layer provided in the microfluidic detection device in FIG. 1;

FIG. 5 is a projection view illustrating the microfluidic detection device in FIG. 1;

FIG. 6 is an enlarged view illustrating a first channel provided in the middle layer of the microfluidic detection device in FIG. 4;

FIG. 7 is an enlarged view illustrating a second glass fiber seating part provided in the middle layer of the microfluidic detection device in FIG. 4;

FIG. 8 is a cross-sectional view illustrating a sensing part provided in the microfluidic detection device in FIG. 4;

FIG. 9 is a schematic view illustrating a combination of a stop valve and the sensing part that are provided in the upper layer and the middle layer of the microfluidic detection device in FIG. 1;

FIG. 10 is an enlarged view illustrating the sensing part provided in the middle layer of the microfluidic detection device in FIG. 4;

FIG. 11 is a perspective view illustrating a lower layer provided in the microfluidic detection device in FIG. 1; and

FIG. 12 shows photographs which are illustrating a bio-marker detected by the sensing part and which are provided for describing an effect of the microfluidic detection device.

### DETAILED DESCRIPTION

Hereinafter, the present disclosure will be described in detail with reference to the accompanying drawings. However, the present disclosure may be implemented in many different forms and is not limited to the embodiments to be described herein. In addition, in order to clearly describe the present disclosure with reference to the drawings, parts irrelevant to the description are omitted, and similar reference numerals denote similar parts throughout the specification.

Throughout the specification, when a part is referred to as being “connected” (connect, contact, combine) to another part, it includes being “directly connected” to another part and “indirectly connected” to another part with still another part disposed therebetween. In addition, unless explicitly described to the contrary, the word “comprise” and variations such as “comprises” or “comprising” will be understood to imply the inclusion of stated elements but not the exclusion of any other elements.

The terminology used herein is for the purpose of describing particular embodiments only and is not intended to limit the present disclosure. As used herein, the singular forms are intended to include the plural forms as well, unless the context clearly indicates otherwise. In addition, it will be further understood that the terms “comprise”, “include”, “have”, and so on when used in the present application, specify the presence or absence of stated features, integers, steps, operations, elements, components, and/or combinations of them but do not preclude the possibility of the presence or addition of one or more other features, integers, steps, operations, elements, components, and/or combinations thereof.

Hereinafter embodiments of the present disclosure will be described in detail with reference to the accompanying drawings.

The present disclosure relates to a microfluidic detection device, and relates to a detection device configured to couple a detection probe and a capture probe to a fluid sample, the detection device being configured to detect a bio-marker included in the fluid sample with a magnet. Particularly, a device configured to allow a fluid sample to be moved in a constant flow by the capillary phenomenon, configured to be structurally provided with a space for collecting bubbles that interfere with the flow of the fluid, and capable of detecting a bio-marker for accurate data reading by washing a biomolecule that is not coupled to a probe may be provided.

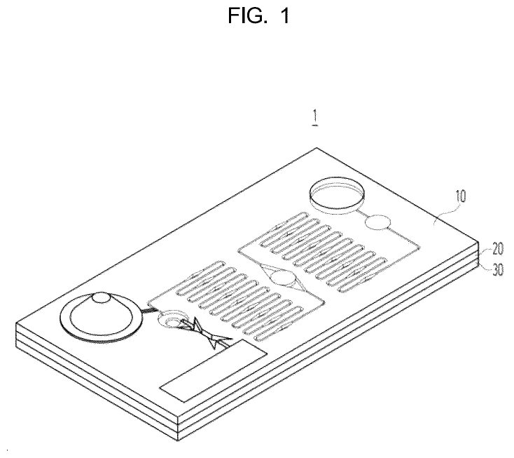

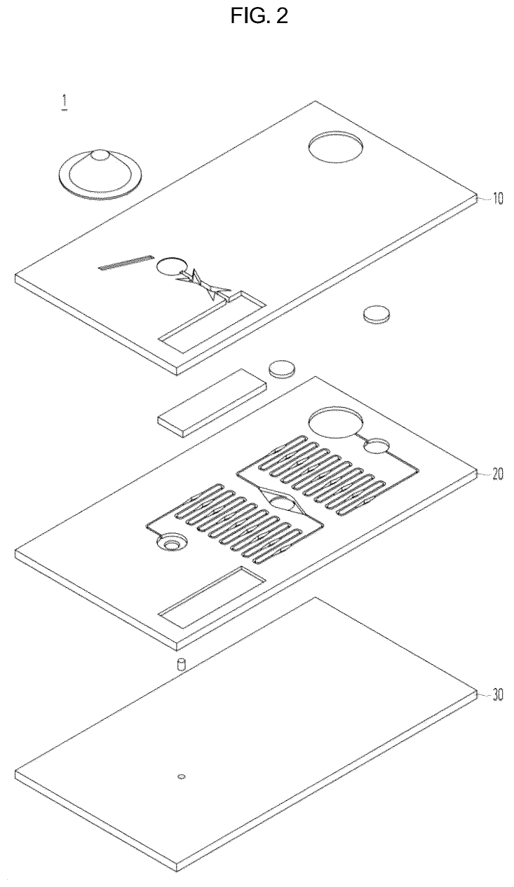

FIG. 1 is a perspective view illustrating a microfluidic detection device according to an embodiment of the present disclosure, and FIG. 2 is an exploded perspective view illustrating the microfluidic detection device according to an embodiment of the present disclosure.

Referring to FIG. 1 and FIG. 2, the microfluidic detection device 1 may include an upper layer 10, a middle layer 20, and a lower layer 30.

The upper layer 10, the middle layer 20, and the lower layer 30 provided in the microfluidic detection device 1 may be provided by performing a surface treatment with oxygen plasma on a 3D printed mold. However, a hydrophilic polymer material may be used for the middle layer 20 so as to transfer a fluid sample by using the capillary phenomenon. At this time, the hydrophilic polymer material may include a PDMS-PEO material, but is not limited thereto.

Meanwhile, the microfluidic detection device 1 is formed by sequentially coupling the upper layer 10, the middle layer 20, and the lower layer 30. Furthermore, in forming the microfluidic detection device 1, the upper layer 10 may be coated with APTES (Aminopropyltriethoxysilane), and the middle layer 20 may be coated with GOPTES ((3-Glycidyloxypropy)trimethoxysilane), so that the upper layer 10 and the middle layer 20 may be attached to each other by chemical coupling. The lower layer 30 may be coupled to the middle layer 20 by using an adhesive such as a tape. For a method in which the upper layer 10, the middle layer 20, and the lower layer 30 are coupled to each other, at least one of combinations thereof may be used, but is not limited to the words listed above.

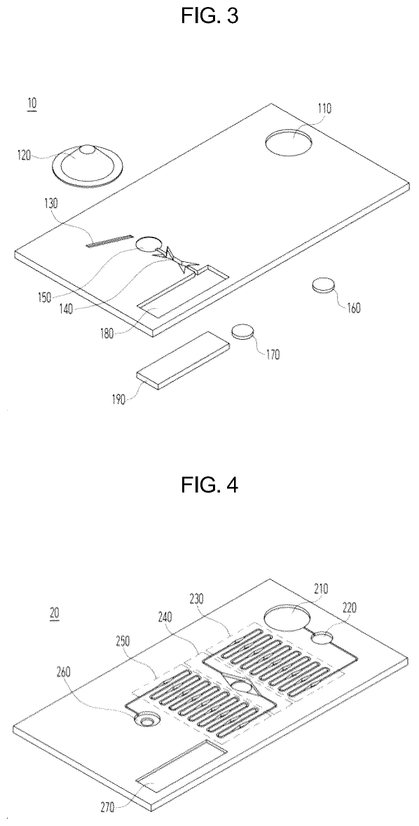

Hereinafter, the microfluidic detection device 1 of the present disclosure will be described in more detail with reference to FIG. 3 to FIG. 11.

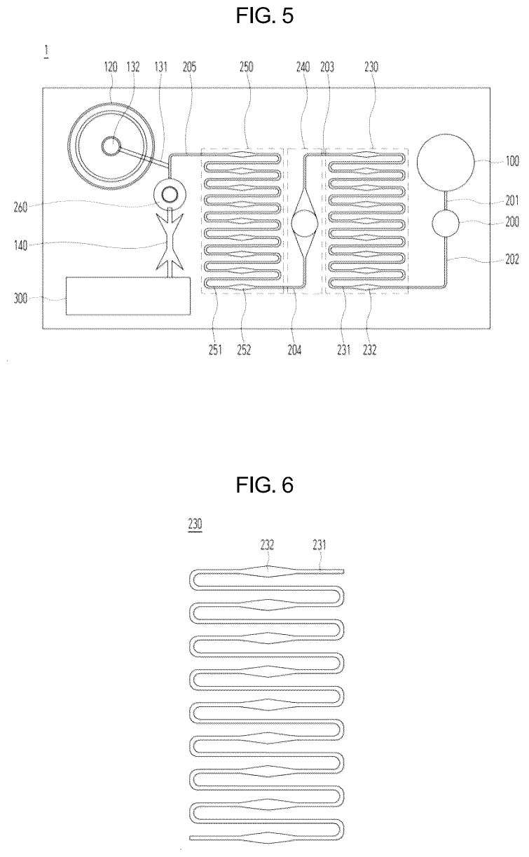

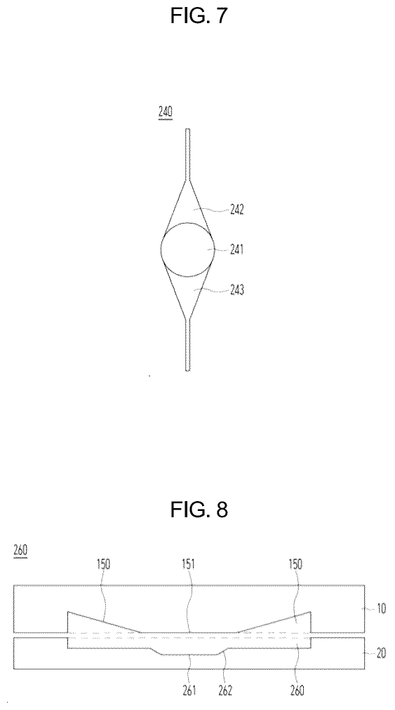

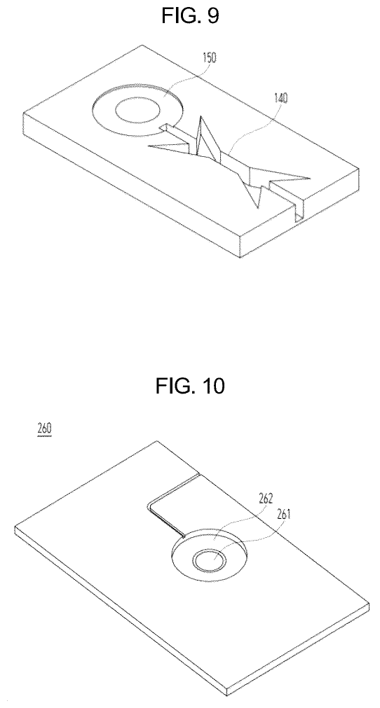

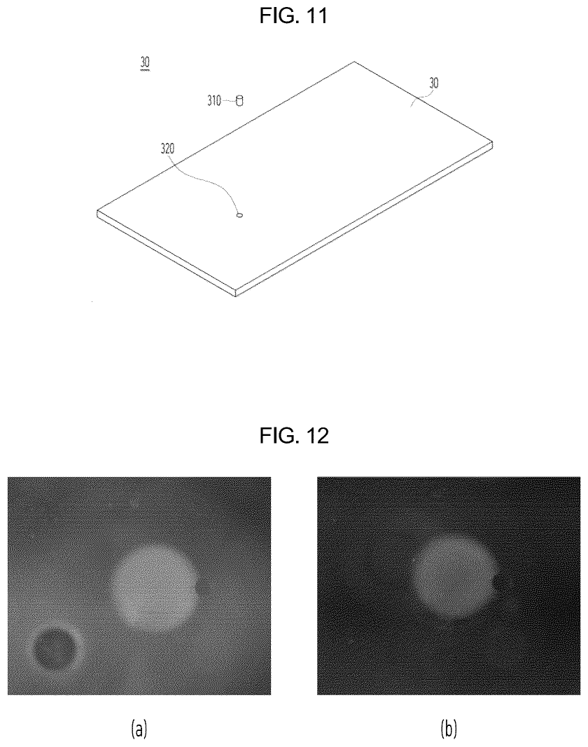

FIG. 3 is a perspective view illustrating an upper layer provided in the microfluidic detection device in FIG. 1, FIG. 4 is a perspective view illustrating a middle layer provided in the microfluidic detection device in FIG. 1, FIG. 5 is a projection view illustrating the microfluidic detection device in FIG. 1, FIG. 6 is an enlarged view illustrating a first channel provided in the middle layer of the microfluidic detection device in FIG. 4, FIG. 7 is an enlarged view illustrating a second glass fiber seating part provided in the middle layer of the microfluidic detection device in FIG. 4, FIG. 8 is a cross-sectional view illustrating a sensing part provided in the microfluidic detection device in FIG. 4, FIG. 9 is a schematic view illustrating a combination of a stop valve and the sensing part that are provided in the upper layer and the middle layer of the microfluidic detection device in FIG. 1, FIG. 10 is an enlarged view illustrating the sensing part provided in the middle layer of the microfluidic detection device in FIG. 4, and FIG. 11 is a perspective view illustrating a lower layer provided in the microfluidic detection device in FIG. 1.

Referring to FIG. 3, the upper layer 10 may include a first inlet part 110, a pouch part 120, an opening and closing part 130, a valve part 140, a bubble collecting part 150, and a first absorption part 180.

The upper layer 10 is capable of transferring a fluid sample to the middle layer 20 by being provided with the first inlet part 110 into which the fluid sample is introduced.

The first inlet part 110 may be positioned at a lower end of a right side of the upper layer 10. The first inlet part 110 may be provided as a circular shape so that the fluid sample is introduced thereinto, and a radius of the first inlet part 110 may be 2.8 mm to 4.2 mm.

In addition, the first inlet part 110 is connected to a second inlet part 210 provided in the middle layer 20, so that the fluid sample is capable of being transferred to the middle layer 20.

The pouch part 120 provided on the upper layer 10 may be a water bag formed in a convex shape, and may be filled with a washing solution inside the pouch part 120. The washing solution may be supplied as the user presses the pouch part 120. Of course, the width and the height of the pouch part 120 may be changed according to the user's needs. However, the pouch part 120 may be provided in a structure in which a negative pressure is not formed. That is, the washing solution is supplied when the pressure is applied to the pouch part 120, but there is a possibility that a backflow of the washing solution may occur when the pressure is removed, so that it is preferable that the pouch part 120 is formed of a material that does not have an elasticity.

However, the pouch part 120 may be controlled such that an appropriate amount of the washing solution in the pouch part 120 is supplied by the opening and closing part 130.

The opening and closing part 130 is a passage provided in the upper layer 10, and is capable of supplying the washing solution provided in the pouch part 120.

The opening and closing part 130 may be provided with a washing flow path 131 and a washing valve 132. The washing flow path 131 may be provided in a shape of a tube having a width of 0.32 mm to 0.48 mm, a height of 0.16 mm to 0.24 mm, and a length of 5.52 mm to 8.28 mm so as to supply the washing solution in the pouch part 120. An upper side of the washing valve 132 is connected to the pouch part 120, the washing valve 132 is provided such that the radius of the washing valve 132 is 0.4 mm to 0.6 mm, and the washing solution in the pouch part 120 is capable of being transferred to the washing flow path 131.

At this time, a speed of the washing solution supplied from the opening and closing part 130 may be controlled by the valve part 140.

The valve part 140 may be provided such that the valve part 140 is capable of stopping the flow of the washing solution for a predetermined time.

In other words, the washing solution is stored in the pouch part 120, the washing solution is passed through the opening and closing part 130, and the flow of the washing solution is stopped by the valve part 140 so that the washing solution is remained.

At this time, the valve part 140 may be provided such that a first side of the valve part 140 is connected to the bubble collecting part 150 and a second side of the valve part 140 is connected to the first absorption part 180.

The valve part 140 is provided such that the height of the valve part 140 is 0.8 mm to 1.2 mm, and the width of the valve part 140 is not particularly limited since the valve part 140 is provided such that bubbles and the washing solution are capable of sufficiently passing through the valve part 140. In addition, a protrusion with an outer angle of 130 degrees may be formed on a first side of the valve part 140 in up and down directions, and a protrusion with an outer angle of 130 degrees may be formed also on a second side of the valve part 140 in the up and down directions. Such protrusions may be formed such that ends of each of the protrusions are directed toward left and right directions of the bubble collecting part 150 and the first absorption part 180.

Such a valve part 140 may be provided such that a washing process is performed while the washing solution and bubbles remain inside the protrusions.

In addition, the bubble collecting part 150 may collect bubbles generated during the washing process. The bubble collecting part 150 is a section in which bubbles included in a complex coupled in the middle layer 20 and bubbles generated during the process of washing the complex are collected, and may separate bubbles that interfere with an immunoassay. At this time, the bubble collecting part 150 may be provided such that the bubble collecting part 150 has a height of 0.09 mm and a radius of 1.0 mm.

In this case, as the washing solution supplied from the pouch part 120 passes through the bubble collecting part 150, the washing process may be performed while a waste solution mixed with impurities is moved to the first absorption part 180.

At this time, the first absorption part 180 may be provided on the first side of the valve part 140 so as to absorb the waste solution generated during the washing process. For example, the waste solution may refer to impurities contained in a used washing solution, bubbles, or a washing solution.

In addition, the first absorption part 180 may be provided such that the washing solution does not backflow to a front end of the first absorption part 180 when the washing solution is oversupplied. In addition, an inner portion of the first absorption part 180 is provided with a concave groove having a lower end thereof perforated so that an absorption pad 190 is provided in the inner portion of the first absorption part 180, and the absorption pad 190 may be inserted between the upper layer 10 and the middle layer 20.

In addition, a first glass fiber 160 and a second glass fiber 170 may be provided between the upper layer 10 and the middle layer 20.

The first glass fiber 160 may be formed of a detection probe bonded to a label, and the second glass fiber 170 may be formed of a capture probe bonded to a magnetic bead. The first glass fiber 160 and the second glass fiber 170 are provided with glass fiber, and a size of pores of the glass fiber is large so that the magnetic bead is easily moved and the fluid sample is capable of smoothly passing therethrough.

More particularly, a detection probe DP in which a probe and a quantum dot QD are coupled to each other may be loaded in the first glass fiber 160, and a capture probe CP coupled to a magnetic bead may be loaded in the second glass fiber 170.

At this time, each of the first glass fiber 160 and the second glass fiber 170 may have a radius of 1.2 mm to 1.8 mm.

Referring to FIG. 4 and FIG. 5, the middle layer 20 may be disposed below the upper layer 10, and may be provided with a plurality of channels that forms a complex by coupling the fluid sample to a probe.

The middle layer 20 may include a transferring part 200, a second inlet part 210, a first loading part 220, a first channel part 230, a second loading part 240, a second channel part 250, a sensing part 260, and a second absorption part 270.

The fluid sample introduced from the second inlet part 210 may be moved through a plurality of transferring parts 200. At this time, the transferring part 200 may transfer the fluid sample and the complex by the capillary phenomenon.

In addition, the second inlet part 210 is interlocked with the first inlet part 110 and is configured to introduce the fluid sample, and may have a radius of 2.8 mm to 4.2 mm, which is the same as the first inlet part 110.

The second inlet part 210 and the first loading part 220 may be connected to each other through a first flow path 201. The first flow path 201 may be provided such that the first flow path 201 has a width of 0.16 mm to 0.24 mm and a height of 0.2 mm to 0.3 mm so that the fluid sample is capable of being moved therethrough.

In order for the fluid sample passing through the first flow path 201 to be mixed with the probe in the first loading part 220, the first glass fiber 160 may be seated in the first loading part 220.

The first loading part 220 has a shape corresponding to the shape of the first glass fiber 160 so that the first glass fiber 160 is stably seated in the first loading part 220, and may have a radius of 1.28 mm to 1.92 mm.

The fluid sample passed through the first loading part 220 may be transferred to the first channel part 230 through a second flow path 202 so that the fluid sample passed through the first loading part 220 forms a first complex.

At this time, the first channel part 230 may form the first complex that is a detection probe-quantum dot complex formed by bonding a bio-marker molecule included in the fluid sample, the quantum dot QD, and the detection probe DP to each other.

In addition, a shape of a flow path in the first channel part 230 may be changed such that the mixing efficiency of the first complex is increased in the first channel part 230.

Referring to FIG. 6, the first channel part 230 may include a first coupling region 231 and a first acceleration region 232.

More particularly, the first channel part 230 is provided with a plurality of layers as the first coupling region 231 is curved and continued, and a plurality of first acceleration regions 232 is provided between a plurality of curved regions, so that the first coupling region 231 may be provided in a form in which the first coupling region 231 is divided such that a plurality of first coupling regions 231 is provided.

The first coupling region 231 may be configured to maintain a latent time for forming the first complex.

At this time, the first coupling region 231 has a width of 0.16 mm to 0.24 mm and a height of 0.2 mm to 0.3 mm, and the first coupling region 231 is capable of transferring the first complex by the capillary phenomenon similar to the plurality of transferring parts 200 provided in the microfluidic detection device 1.

When particles exist on a fluid interface, an interface transformation around the particles occurs and a surface free energy increases, and the capillary phenomenon may refer to a force that is a driving force in a phenomenon in which the particles are arranged and moved in a direction in which the total surface area of the fluid interface is reduced except for an area occupied by the particles. By such a driving force, the microfluidic detection device 1 may be driven without an external power device.

By such a driving force, the first acceleration region 232 may generate an acceleration in the flow of the first complex, so that a mixing speed of the first complex may be increased.

At this time, the first acceleration region 232 formed in a rhombus shape is provided in the middle of the first coupling region 231, and expands to a width of 0.56 mm to 0.84 mm. Furthermore, a length of the first acceleration region 232 may be changed according to the user's choice.

The first coupling region 231 and the first acceleration region 232 are configured such that a plurality of expanded regions is provided so that contraction and expansion are repeatedly performed so as to increase the mixing efficiency. Accordingly, the capture probe CP and the fluid sample may be appropriately mixed.

In other words, the fluid sample and the detection probe DP are passing through the first coupling region 231 while maintaining a constant latent time, and a vortex for mixing is generated in the first acceleration region 232, so that the fluid sample and the detection probe DP may be coupled to each other as the degree of mixing for the first complex increased.

Therefore, in the first acceleration region 232, a diffusion time of the bio-marker molecule included in the fluid sample and the detection probe DP inside the first complex may be provided.

The first complex that passes through the plurality of first acceleration regions 232 may be transferred from the first channel part 230 to the second loading part 240 through a third flow path 203. The third flow path 203 connects the first channel part 230 and the second loading part 240, and may have a width of 0.16 mm to 0.24 mm and a height of 0.2 mm to 0.3 mm.

The second loading part 240 may be provided such that the second glass fiber 170 is seated in the second loading part 240.

Referring to FIG. 7, the second loading part 240 may include a glass fiber seating region 241, an expansion region 242, and a mixing region 243.

The second loading part 240 may be provided such that the second loading part 240 mixes the capture probe CP in the second glass fiber 170 with the first complex.

The glass fiber seating region 241 has a shape corresponding to the shape of the second glass fiber 170 so that the second glass fiber 170 is seated in the glass fiber seating region 241, and may have a radius of 1.28 mm to 1.92 mm.

The first complex is supplied to a first side of the glass fiber seating region 241, and the first side of the glass fiber seating region 241 may be provided with the expansion region 242 so as to adjust the flow of the first complex.

A first side of the expansion region 242 is connected to the third flow path 203, and may receive the first complex.

In addition, the expansion region 242 may be provided with a structure in which the width of the expansion region 242 increases from the third flow path 203 to the glass fiber seating region 241. More particularly, the maximum width of the expansion region 242 is 2.4 mm to 3.6 mm, and the angle at which the expansion region 242 meets the circumference of the glass fiber seating region 241 is set to 158 degrees on each side.

Due to the structure of the expansion region 242, the speed of the first complex is reduced, so that the first complex may remain in the glass fiber seating region 241 and the second glass fiber 170 may absorb the first complex.

However, a momentary vortex may occur in the expansion region 242, so that bubbles may occur inside the first complex. In this situation, a partial gas aggregation may occur at an edge where expansion region 242 and the glass fiber seating region 241 are in contact with each other.

The mixing region 243 may be provided on a second side of the glass fiber seating region 241.

The mixing region 243 may be provided on the second side of the glass fiber seating region 241, and may form a second complex by mixing the first complex with the capture probe CP.

The mixing region 243 may be provided in a shape symmetrical with the expansion region 242 with respect to the center of the glass fiber seating region 241. For example, a section having a shape that gradually narrows from both outer sides of the glass fiber seating region 241 may be formed.

At this time, the mixing region 243 is connected to a fourth flow path 204, and the width of the mixing region 243 may be narrowed as the mixing region 243 is connected to the fourth flow path 204. Since the area of the mixing region 243 having the shape describe above is gradually narrowed, the mixing of the first complex and the capture probe may be accelerated.

The fourth flow path 204 connects the mixing region 243 of the second loading part 240 and the second channel part 250 to each other, and may have a width of 0.16 mm to 0.24 mm and a height of 0.2 mm to 0.3 mm.

The fourth flow path 204 may transfer the mixture of the first complex and the capture probe CP to the second channel part 250.

The second channel part 250 may form the second complex by coupling the first complex and the capture probe CP in the second glass fiber 170 to each other.

The second complex may be formed as a capture probe-magnetic bead complex (MBs-Ab, Magnetic Beads-Antibody) formed by coupling the magnetic bead to the capture probe CP.

In addition, the second channel part 250 may include a second coupling region 251 and a second acceleration region 252.

The second coupling region 251 may be configured to maintain a latent time for realizing the high mixing degree of the second complex, and the second acceleration region 252 may provide a diffusion time to the magnetic bead and the capture probe CP of the second complex.

The second complex formed in the second channel part 250 may be configured as a DP-target-CP complex.

However, since the shape and configuration of the second coupling region 251 and the second acceleration region 252 of the second channel part 250 are the same as that of the first coupling region 231 and the first acceleration region 232 of the first channel part 230, the detailed description thereof will be omitted.

The second complex may be transferred to the sensing part 260 through a fifth transferring path 205.

The fifth transferring path 205 connects the second channel part 250 and the sensing part 260 to each other, and may have a width of 0.2 mm and a height of 0.25 mm.

Meanwhile, the fifth transferring path 205 may be connected to the opening and closing part 130 at a point close to the sensing part 260. At this time, a first side of the opening and closing part 130 may be connected to the fifth transferring path 205, and a second side of the opening and closing part 130 may be connected to the pouch part 120, thereby being capable of supplying the washing solution.

As a result, the fifth transferring path 205 may transfer the second complex. That is, the fifth transferring path 205 may supply the second complex mixed with the washing solution as the fifth transferring path 205 passing through the opening and closing part 130 and is positioned close to the sensing part 260.

The sensing part 260 may detect the magnetic bead contained in the second complex.

Referring to FIG. 8 to FIG. 10, the upper side of the sensing part 260 may be coupled to the bubble collecting part 150, and the sensing part 260 may include a sensing region 261 and a washing region 262.

A magnet may be coupled to a lower side of the sensing part 260 so that the sensing part 260 is capable of sensing the magnetic bead.

The sensing part 260 may be provided such that the sensing part 260 detects a bio-marker contained in the second complex and collects bubbles formed during washing. At this time, the sensing part 260 may be provided in an upper surface of the middle layer 20, and may have a radius of 1.6 mm to 2.4 mm. The sensing region 261 and the washing region 262 may be provided on a surface of the sensing part 260 with a predetermined depth.

More particularly, the sensing region 261 is provided on a lower end of the sensing part 260, and may detect the magnetic bead contained in the second complex. The sensing region 261 may have a radius of 0.75 mm so as to capture the magnetic bead.

In other words, the sensing part 260 is a region detecting the DP-target-CP complex which is the second complex. That is, the DP-target-CP complex is detected at the lower side of the sensing part 260 by reacting with the magnet positioned at the lower side of the magnet due to the magnetic bead contained in the DP-target-CP complex. Due to this process, the bio-marker contained in the second complex is capable of being detected at the sensing part 260.

The washing region 262 may be positioned at an upper side of the sensing region 261, and may be formed as an inclined surface that is gradually widened from the sensing region 261 to the washing region 262 so as to wash the second complex. The washing region 262 may have a radius of 0.72 mm to 1.08 mm and a depth of 0.06 mm to 0.08 mm so that the second complex may flow to the sensing region 261.

In the sensing part 260, the sensing region 261 and the washing region 262 may have concave shapes in which the widths thereof are narrowed toward the sensing region 261. This structure of the sensing section 260 provides resistance to the magnetic bead so that the magnetic bead is not damaged by the inflow of washing solution.

In addition, since the washing solution input into the sensing part 260 is supplied by the user's operation of the pouch part 120, the second complex is preferentially introduced into the sensing part 260, and the washing solution is capable of being input by the user's operation.

In addition, the washing solution introduced into the sensing part 260 may be provided such that the waste solution is floated and separated so that the bio-marker is efficiently detected in the sensing region 261. The waste solution may include the used washing solution, and the detection probe DP and biomolecules that are not coupled to the second complex.

In addition, bubbles generated in the sensing part 260 is floated upward, and may be collected in the bubble collecting part 150 provided above the sensing part 260.

In other words, the sensing part 260 may allow the waste solution and bubbles excluding the bio-marker to float upward, and may transfer the waste solution and the bubbles to the first absorption part 180 connected to the valve part 140.

At this time, while the washing is performed in the sensing part 260, the washing solution may be controlled such that the washing solution remains in the valve part 140.

At this time, the second absorption part 270 may be provided at a first side of the middle layer 20 so as to prevent the washing solution from backflowing to the sensing part 260.

The second absorption part 270 may have a concave shape such that the absorption pad 190 is seated inside the second absorption part 270.

More particularly, the second absorption part 270 may form an absorption region 300 which is bonded to the first absorption part 180 and which accommodates the absorption pad 190 therein. At this time, the absorption region 300 may be formed such that the absorption region 300 has a width of 3.6 mm to 5.4 mm, a length of 12 mm to 18 mm, and a height of 1.28 mm to 1.92 mm.

In addition, the absorption pad 190 accommodated inside the absorption region 300 may be provided in a size smaller than that of the specification of the absorption region 300. The absorption pad 190 may be formed of synthetic resin such as non-woven fabric having pores therein for smooth absorption. As the absorption pad 190, at least one selected from a common non-woven fabric material such as polypropylene, polyethylene, polyester, acrylic-based synthetic resin, Teflon, polyvinyl chloride resin, polypropylene, polyethylene terephthalate, polyvinylidene fluoride, polytetrafluoroethylene, and a copolymer thereof, natural fiber, artificial fiber, synthetic fiber, glass fiber, and a combination thereof may be used, but is not limited to the words listed above.

Meanwhile, the absorption region 300 is formed between the upper layer 10 and the middle layer 20, and the middle layer 20 may be connected to the upper layer 10 by chemical coupling.

A lower layer 30 is disposed below the middle layer 20, and a tape is attached to an upper portion of the lower layer 30, so that the lower layer 30 is capable of being coupled to the middle layer 20.

Referring to FIG. 11, the lower layer 30 may be provided with a magnet 310 and a magnet seating part 320.

The magnet 310 may have a width and length of 0.8 mm to 1.2 mm, and the magnet seating part 320 may be provided with a concave groove having a radius of 0.4 mm to 0.6 mm and a height of 0.8 mm to 1.2 mm so that the magnet 310 is accommodated in the magnetic seating part 320.

As the magnet 310 is accommodated inside the magnet seating part 320 and the lower layer 30 including the magnet seating part 320 is attached to the middle layer 20, the bio-marker is capable of being detected at the sensing part 260 positioned above the magnet 310.

FIG. 12 shows photographs enlarging and illustrating the sensing part so as to describe an effect of the microfluidic detection device.

FIG. 12A is a photograph enlarging and illustrating a bio-marker detected in the sensing region 261 before the washing solution is supplied, and FIG. 12B is a photograph enlarging and illustrating the bio-marker detected in the sensing region 261 after the washing solution is supplied.

As the washing solution is supplied to the sensing part 260, the detection probe DP and biomolecules that are not coupled to the second complex are washed along with the washing solution, so that the bio-marker is capable of being efficiently detected in the sensing part 260.

In the microfluidic detection device 1 according to an embodiment of the present disclosure, the fluid sample flows by the capillary phenomenon, the space for collecting bubbles that interfere with the flow of the fluid is structurally provided, and the bio-marker is capable of being detected by washing the detection probe-quantum dot complex that is not coupled to the bio-marker.

In an immunodetection method for measuring a fluorescence intensity by irradiating a quantum dot contained in the detection probe DP with an LED light source to induce fluorescence and then quantifying and detecting a target concentration in an immunoassay reaction, such a microfluidic detection device 1 detects the bio-marker on which impurities are removed, a fluorescence signal of impurities is reduced and only a fluorescence signal of the bio-marker is detected, so that there is an effect that the accuracy in detecting the fluorescence intensity is increased.

The above descriptions on the present disclosure are for illustration, and those skilled in the art to which the present disclosure pertains may understand that the descriptions may be easily modified into other specific forms without changing the technical idea or essential features of the present disclosure. Therefore, it should be understood that the embodiments described above are illustrative in all respects and not restrictive. For example, each component described as a single type may be implemented in a dispersed form, and likewise components described as distributed may be implemented in a combined form.

The scope of the present disclosure is indicated by the following claims rather than the above detailed description, and all changes or modifications derived from the meaning and scope of the claims and their equivalents should be interpreted as being included in the scope of the present disclosure.

What is claimed is:

1. A microfluidic detection device in which microfluid flows by a capillary phenomenon, the microfluidic detection device comprising:
an upper layer provided with a first inlet part configured to supply a fluid sample;
a middle layer disposed below the upper layer and provided with a second inlet part interlocked with the first inlet part, the middle layer being provided with a plurality of channels forming at least one complex by coupling the fluid sample to each probe; and
a lower layer disposed below the middle layer and provided with a magnet seating part supported by the middle layer and a magnet.

2. The microfluidic detection device of claim 1, further comprising:
a first glass fiber, a second glass fiber, and an absorption pad that are disposed between the upper layer and the middle layer,
wherein a detection probe formed by coupling the probe to a label is loaded in the first glass fiber, and
wherein a capture probe formed by coupling the probe to a magnetic bead is loaded in the second glass fiber.

3. The microfluidic detection device of claim 2, wherein the upper layer comprises:
a pouch part filled with a washing solution;
an opening and closing part connected to the pouch part and configured to supply the washing solution;
a bubble collecting part configured to capture bubbles contained in the complex;
a valve part provided on a first side of the bubble collecting part and configured to stop a flow of the washing solution for a predetermined time; and
a first absorption part connected to the valve part and provided in a shape that surrounds the absorption pad, thereby absorbing a waste solution,
wherein the bubble collecting part has a polygonal shape including a circular shape, and is provided in a shape in which a height thereof decreases from an outer portion of the polygonal shape to a center of the polygonal shape, thereby collecting the bubbles.

4. The microfluidic detection device of claim 3, wherein the middle layer comprises:
a transferring part comprising a plurality of transferring parts provided so as to transfer the fluid sample and the complex;
a first loading part in which the first glass fiber is seated, the first loading part being configured to load the detection probe;
a first channel part in which a first complex is formed by coupling a bio-marker molecule contained in the fluid sample to the detection probe;
a second loading part in which the second glass fiber is seated, the second loading part being configured to load the capture probe;
a second channel part in which a second complex is formed by coupling the first complex to the capture probe;
a sensing part configured to concentrate the magnetic bead contained in the second complex; and
a second absorption part formed in a concave shape such that the absorption pad is seated therein, the second absorption part being configured to absorb the waste solution.

5. The microfluidic detection device of claim 4, wherein the first channel part comprises:
a first coupling region comprising a plurality of first coupling regions so as to maintain a latent time for mixing the first complex; and
a first acceleration region comprising a plurality of first acceleration regions provided as expanded regions in the first coupling regions, the first acceleration region being configured to provide a diffusion time to the bio-marker molecule and the detection body contained in the fluid sample of the first complex.

6. The microfluidic detection device of claim 4, wherein the second loading part comprises:
a glass fiber seating region formed in a shape corresponding to that of the second glass fiber and in which the second glass fiber is seated;
an expansion region provided on a first side of the glass fiber seating region and configured to control a flow of the first complex; and
a mixing region provided on a second side of the glass fiber seating region and configured to accelerate mixing of the first complex and the capture probe.

7. The microfluidic detection device of claim 4, wherein the second channel part comprises:
a second coupling region comprising a plurality of second coupling regions so as to maintain a latent time for forming the second complex; and
a second acceleration region comprising a plurality of second acceleration regions provided as expanded regions in the second coupling region, the second acceleration region being configured to provide a diffusion time to the first complex and the capture probe so that the second complex is formed.

8. The microfluidic detection device of claim 7, wherein the sensing part comprises:
a sensing region configured to concentrate and sense the magnetic bead contained in the second complex by being in contact with the magnet provided below the sensing region; and
a washing region provided slantly such that the washing region is widened upward from the sensing region, the washing region being configured to provide a resistance force to the magnetic bead so that the magnetic bead is prevented from being washed out.

9. The microfluidic detection device of claim 8, wherein the sensing region is configured to detect a bio-marker contained in the second complex.

10. The microfluidic detection device of claim 4, wherein the transferring part comprises:
a first flow path connecting the second inlet part and the first loading part to each other;
a second flow path connecting the first loading part and the first channel part to each other;
a third flow path connecting the first channel part and the second loading part to each other,
a fourth flow path connecting the second loading part and the second channel part to each other; and
a fifth transferring path connecting the second channel part and the sensing part to each other.

11. The microfluidic detection device of claim 10, wherein the transferring part is configured to transfer the fluid sample and at least one of the complex by the capillary phenomenon.

12. The microfluidic detection device of claim 4, wherein an absorption region in which the absorption pad is accommodated is formed by bonding the first absorption part and the second absorption part to each other.

13. The microfluidic detection device of claim 12, wherein the absorption region is configured to absorb the washing solution so that the washing solution is prevented from backflowing to the sensing part.
